(19) United States
(12) Patent Application Publication
(10) Pub. No.: US 2008/0050795 A1
(43) Pub. Date: Feb. 28, 2008

(54) Mixture Of At Least Two Different Antibodies Specific For Predetermined Antigens And Use Of The Mixture

(75) Inventors: Per Mansson, Sollentuna (SE); Daniel Berggren, Jarfalla (SE); Carl Lundberg, Stockholm (SE); Ann-Charlotte Hellgren, Lidingo (SE); Lena Hojvall, Jarfalla (SE)

Correspondence Address: BACON & THOMAS, PLLC, 625 SLATERS LANE, FOURTH FLOOR, ALEXANDRIA, VA, 22314, US

(73) Assignee: Biosensor Applications Sweden AB, Sundbyberg (SE)

(21) Appl. No.: 10/580,232

(22) PCT Filed: Nov. 19, 2004
(86) PCT No.: PCT/SE04/01697

§ 371 Date: Feb. 7, 2007

Related U.S. Application Data
(60) Provisional application No. 60/523,307, filed on Nov. 20, 2003.

(51) Int. Cl. C07C 211/00; C07C 331/00; C07D 221/22; C07D 251/00; C07D 257/08; C07D 317/44; C07D 451/00; C07F 15/00; C07H 19/00; C07H 21/02; C07K 14/00; C07K 16/00; C07K 2/00; C12M 3/00; C12N 1/00; C12N 1/12; C12N 1/20; C12N 7/00; C12P 21/08

(52) U.S. Cl. 435/183; 435/235.1; 435/252.1; 435/253.1; 435/254.1; 435/287.1; 435/287.2; 435/325; 530/300; 530/350; 530/387.1; 530/388.1; 530/389.1; 536/22.1; 536/23.1; 544/179; 544/180; 546/130; 546/74; 549/440; 558/485; 558/486; 564/305; 564/441; 568/300

(57) ABSTRACT

A mixture of isolated or synthetic affinity molecules in a liquid carrier is disclosed. The mixture comprises at least two different affinity molecules, each with affinity for a predetermined analyte, for use in a single or multi flow cell piezoelectric crystal micro balance apparatus. Each isolated or synthetic affinity molecule forms together with the predetermined analyte an interaction pair selected from the group consisting of anion-cation, antibody-antigen, receptor-ligand, enzyme-substrate, oligonucleotide-oligonucleotide with complementary sequence, oligonucleotide-protein, oligonucleotide-cell, and peptide nucleic acid (PNA) oligomer-polynucleotide, wherein the polynucleotide may be selected from the group consisting of RNA, DNA and PNA polymers complementary to PNA oligomer. Use of the mixture for introduction into the liquid flow of a single or multi flow cell piezoelectric crystal micro balance apparatus is also described, as well as a kit containing the mixture.

The present invention relates to a mixture of at least two different affinity molecules, each specific for a predetermined analyte and use of the mixture. More precisely, the mixture of the invention is a mixture of isolated or synthetic affinity molecules in a liquid carrier comprising at least two different affinity molecules, each with affinity for a predetermined analyte, for use in a single or multi flow cell piezoelectric crystal micro balance apparatus.

### BACKGROUND

The piezoelectric technique is based on the well-known principle of measuring the mass change in real time by measuring the frequency of a piezoelectric quartz crystal. The piezoelectric crystal device consists of a quartz crystal wafer having a metal electrode on both sides. These electrodes are used to induce an oscillating resonant frequency, that is dependent on the mass of the electrode and a change of the frequency is directly related to the change of the deposited mass on the electrode. Piezoelectric crystals can therefore be used for sensitive mass measurement and are therefore called Quartz Crystal Microbalances (QCM). A number of equations have been proposed to describe the relationship between frequency changes and mass deposition of the crystal.

There is a large number of patents directed to the detection of a predetermined chemical or biomolecule in a solution by use of a piezoelectric crystal microbalance, for example, U.S. Pat. Nos. 4,735,906, 4,789,804, and 5,705,399. When the presence of several individual chemicals or biomolecules (analytes) are to be determined in the same test solution, then it is advantageous to have a system that can simultaneously handle a plurality of microbalances with flow cells which are individually specific for one of the analytes to be detected. Such a system has been disclosed in our International patent application WO2004001392, and it is particularly useful for screening of a large number of samples in a short time period.

The mixture of the present invention can be used in the piezoelectric crystal microbalance devices that are disclosed in the above mentioned patents and patent application.

### DESCRIPTION OF THE INVENTION

The present invention provides a mixture of isolated or synthetic affinity molecules in a liquid carrier comprising at least two different affinity molecules, each with affinity for a predetermined analyte, for use in a single or multi flow cell piezoelectric crystal micro balance apparatus.

The mixture of the invention is preferably such that each isolated or synthetic affinity molecule forms together with the predetermined analyte an interaction pair selected from the group consisting of anion-cation, antibody-antigen, receptor-ligand, enzyme-substrate, oligonucleotide-oligonucleotide with complementary sequence, oligonucleotide-protein, oligonucleotide-cell, and peptide nucleic acid (PNA) oligomer-polynucleotide, wherein the polynucleotide may be selected from the group consisting of RNA, DNA and PNA polymers complementary to the PNA oligomer.

It should be understood that affinity molecules are individually produced in any suitable way, such as by isolation from a natural or synthetic source, by use of chemical or biological synthesis or both, by cleavage from a larger molecule etc.

In a preferred embodiment of the invention each isolated or synthetic affinity molecule is selected from the group consisting of monospecific polyclonal or monoclonal antibodies, antibody fragments or derivatives thereof each with affinity for a predetermined analyte, i.e. a predetermined antigen.

The antibodies can be custom made by specialized producers, bought from different suppliers or synthesized by procedures known from the literature such as from Hybridoma Technology in the Biosciences&Medicine. T. A. Springer, editor, Plenum Press, 1985.

In a presently preferred embodiment of the mixture of the invention the concentration of each of the different affinity molecules is between 0.01-0.8 mg/ml of the liquid carrier.

The liquid carrier is exemplified by water and it may additionally contain a buffer, stabilizers and/or preservatives, and can be selected based on the composed mixture of choice by a man of ordinary skill in the art. The stabilizer can e.g. be a mixture of surfactants (e.g. Tween® 20 or Tween® 80 or similar) and/or various proteins (e.g. albumin, casein or other protective agents or blocking agents).

Examples of individual analytes that can be detected in a test solution by use of the mixture of the invention in a single or multi flow cell piezoelectric crystal micro balance apparatus are different narcotics such as cocaine, heroin, amphetamine, methamphetamine, cannabinols, tetrahydrocannabinols (THC), and methylenedioxy-N-methylamphetamine (ecstacy), different explosives such as trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG), and different biomolecules, microorganisms and parts thereof. Examples of microorganisms bacteria, bacterial spores, mycobacteria, fungi, and viruses.

Another aspect of the invention is directed to the use of a mixture according to the invention for introduction into the liquid flow of a single or multi flow cell piezoelectric crystal micro balance apparatus.

There are two slightly different concepts of using a piezoelectric crystal micro balance apparatus that are of preferred interest in the present invention, the competition mode and the displacement mode.

In one embodiment of the use of the mixture according to the invention, the mixture of affinity molecules is mixed with a test sample solution that possibly contains one or several of the predetermined analyte(s) prior to introduction into the liquid flow of the apparatus. The free analyte molecule(s) in the test sample compete for the affinity molecules with analyte analogue(s) that is (are) immobilized on the electrode surface(s) of the piezoelectric crystal micro balance apparatus and the change in mass of the electrode is registered. This use exemplifies the competition mode concept.

In another embodiment of the use of the mixture according to the invention, the introduction into the liquid flow of the apparatus is for activation or reactivation of one or several flow cell crystal electrode(s) by attachment to analyte-analogues of the predetermined analytes which analyte-analogues are immobilized on the electrode(s). This use is adapted for the displacement mode concept.

The affinity molecules attach to the analyte-analogues on the electrode surfaces but weaker than to the analyte in question, so when the analyte is present in a test solution, the analyte forms an interaction pair with the affinity molecule, which is detached from the analyte-analogue and the weight loss of the electrode is registered, indicating the presence of the analyte in the test solution.

The mixture of the invention may be introduced into the continuous flow of the apparatus at intervals, for example at interval selected from the range of 20 minutes to 24 hours, e g every 30 minutes.

The mixture of the invention may also be introduced into the continuous flow of the apparatus after recovery of the electrode, i.e. removal of antibodies from the electrode surface, with an pH-lowering agent, such as glycine.

The mixture of the invention is either inherently stable or is stabilized by addition of a stabilizer such as a protein, e g albumin.

An additional aspect of the invention is directed to a kit containing a stable or stabilized mixture according to the invention.

The invention will now be further by embodiments where the affinity molecules are antibodies and the analytes are antigens.

In the competition mode, the crystals in the multi flow cell piezoelectric balance apparatus disclosed in our International patent application WO2004001392, are first surface modified with the antigen-analogues as described above prior to exposure of a test sample solution containing unknown amounts of analytes, i.e. antigens, and the mixture of predetermined amounts of specific antibodies. The immobilized and free analyte molecules now compete for the binding to the antibodies. When this mixture of test sample and antibodies is exposed to the surface modified crystal surface, the increase in the mass of the crystal, i.e. the decrease of frequency, is inversely related to the analyte concentration in the test sample.

The mixture of the invention is in particular useful for activation or reactivation of piezoelectric crystal microbalance flow cells that have at least two different antigen-analogues attached to or coated on, i e immobilized on, an electrode in one cell or separate electrodes in separate cells.

It should be understood that the expressions “antigen-analogues attached to or coated on or immobilized on an electrode” comprises all kinds of spacer molecules between the antibody-binding antigenic site and the metal surface of the flow cell electrode. Examples of such spacer molecules are comprised by our International patent applications WO2004001417 and WO20041416.

The different antigen analogues of the predetermined antigens, each bind to antibodies that are specific for said antigens to form an immunocomplex. The immunocomplex-coated piezoelectric crystal microbalance flow cells are used as antibody-activated flow cells in a sensor system using displacement mode for detection of at least two different analytes, i. e. predetermined antigens to be detected, in a fluid sample, i e a test solution.

The stable or stabilized mixture of the invention enables rapid analysis of possible presence of narcotics and/or explosives with a piezoelectric crystal microbalance instrument that has one, two or more flow cells comprising at least two different antigen-analogues of predetermined antigens to be detected in a screening situation e.g. at customs or airport passenger control.

The antibodies of the mixture need not be labeled, and therefore the mixture of the invention enables a label-free immunosensor system for detection of narcotic drugs, explosives and other substances described herein. The key feature of the technique is a displacement of antibodies from an immunocomplex-coated piezoelectric crystal due to the presence of the substance. The displacement of antibodies is monitored as a change in oscillating characteristics of the crystal, usually as an increase in the frequency of the piezoelectric crystal and is directly related to the concentration of the substance.

The displacement principle is illustrated in FIG. 1. As can be seen in the figure the QCM-electrode is first coated with an immunocomplex of antigen-analogue and antibody. When this immunocomplex-coated electrode is exposed to a liquid sample containing the antigen the soluble antigens compete for the antibodies in the immunocomplex on the surface and causes their displacement with a resulting increase in frequency of the crystal. The extent of displacement is directly related to the concentration of antigen in the liquid sample. However, it is a very delicate task to design the immunocomplex in order to modulate the sensitivity. If the affinity between the surface immobilized antigen-analogue and the antibody is too strong there will be only a slight displacement or no displacement at all. In most cases in other immunoassay techniques it is desirable that the antibody-antigen interaction is very strong and irreversible. However, if the affinity between the antigen-analogue and the antibody is too low in the displacement analysis, the antibody dissociates very easily and the sensitivity can be high during only a limited time period.

### SHORT DESCRIPTION OF THE DRAWINGS

FIG. 1 illustrates the displacement mechanism taking place on a QCM surface.
Note: The representation is not to scale. In reality an antibody is much larger than the drug-molecule analogues, that are bound to the metal, e.g. gold surface. On the left hand side of the figure the drug molecules enter the BioCell®, where antibodies are attached to antigen-analogues on the sensor. On the right hand side, antibodies are released in the presence of the drug (i.e. predetermined antigen) molecules in the solution above the crystal surface. The mass ratio between the drug molecules and the antibody yields a multiplication effect in terms of the mass change.

FIG. 2 is a schematic drawing of the analysis system used in the examples.

FIG. 3 shows the frequency decrease after an injection of about 4 microliters of an antibody mixture containing 0.1 mg/ml of antibodies specific for cocaine, heroin, amphetamine and ecstasy (MDMA), respectively.

FIG. 4 shows when a sample containing 2 ng amphetamine is injected by looping a sample plug into the flow, that is going through the cells. Apart from the positive response in the amphetamine cell in FIG. 4 no responses (cross reactions) were observed in the other cells.

FIG. 5 shows when a mixture containing 2 ng of TNT and 2 ng of cocaine was injected into 4 serially connected cells, and

FIG. 6 shows when about 2 ng of TNT were injected in the same cell configuration.

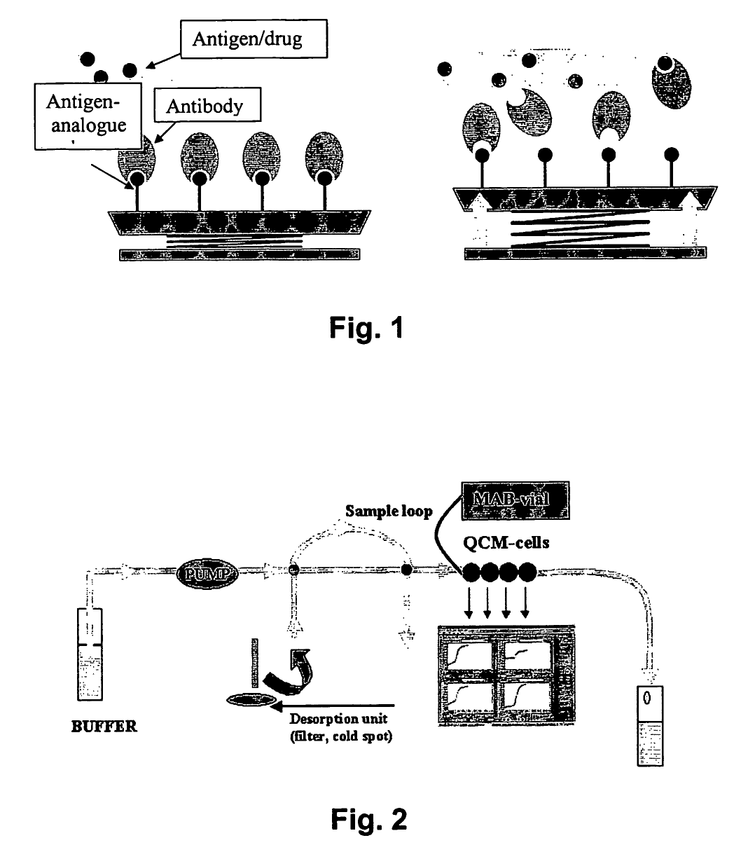

The operation of a QCM instrument disclosed in our co-pending International patent application WO2004001392, the contents of which is hereby included herein by reference, is fully automatic after insertion of the sample to be analyzed on a filter or injection of the sample or samples by other techniques to the QCM-instrument. The automatic operation of the instrument comprises desorption of an analyte from e.g. a filter to a cold spot, extraction with an appropriate buffer following by introduction of the analyte-containing buffer into the antibody-activated QCM-cells for analysis by monitoring the frequency shifts. FIG. 2 show a schematic drawing of the system.

### Electrode Preparation

The QCM-electrodes in the biocells in the analysis system instrument were prepared for displacement reaction according to our co-pending International patent applications WO20041416 and WO20041417. Each of the gold electrodes on the piezoelectric crystals (QCM-crystals) are surface-coated with their respective antigen-analogues that are derivatives of predetermined analyte-antigens that are to be detected. Each coating-antigen analogue has been modified in order to show a weaker affinity to an antibody than the analyte-antigen in solution. The surface modified QCM-crystals were inserted into the cell housing (Biocell) and thereafter docked to the flowing system in the instrument. The eluent (buffer) is pumped through the consecutive cells, which are stabilized within a few minutes.

### A Typical Analysis Run Can Be Described As Follows:

The sample is introduced into the cell in the automatic instrument by looping-in a small volume (a sample plug) of an aqueous solution of the sample to be analyzed (see e.g. the co-pending International patent application WO2004001392).The sample to be tested has usually been collected onto a filter by wiping a suspected surface and/or collection of surrounding vapor by using a vacuum cleaner or pre-concentrator of some kind. The analyte(s) of the collected sample on the filter is transferred and purified by means of a desorption process described in the International patent application WO 03/073070, the contents of which is hereby included herein by reference, or by means of an extraction by using an ionizer probe, such as one described in our co-pending International patent application PCT/SE/000767. A mixture of different monoclonal antibodies (MAB-mixture) against the various analytes, i. e. different antigens, are injected into the various cells prior to the sample plug is introduced into the flow.

Interestingly, wiping of the skin of human drug addicts with a filter, cloth or the like, and analysis performed in accordance with the present invention has given good analysis results for tested narcotics.

The mixture of antigen-specific antibodies according to the invention contains at least two different antibodies, and depending on the number of different antigens that is desired to detect in one run, the mixture contains e.g. 3, 4, 5, 6, 7, 8 or more different antibodies.

Examples of analytes to be detected are different narcotics (antigens) selected from the group consisting of cocaine, heroin, amphetamine, methamphetamine, cannabinols, tetrahydrocannabinols (THC), and methylenedioxy-N-methylamphetamine (ecstacy), and different explosives from the group consisting of trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG).

For example, the mixture of antigen-specific antibodies according to the invention may contain the antibodies used in the each of the FIGS. 2-6. It should be noted that different antibodies specific for explosives can be mixed with different antibodies specific for narcotics, thus enabling detection of both explosives and narcotics in one run.

The volume of the injected MAB-mixture is typically 2-50 microliters and the MAB is injected about a few seconds prior to when the sample is introduced into the QCM-electrode-containing cell by an automatic micro-injection from a MAB-container of some kind, hereinafter exemplified by a vial which is integrated into the instrument (see FIG. 2). MAB-mixture can also be injected at intervals, manually or automatically, in a apparatus with continuous flow.

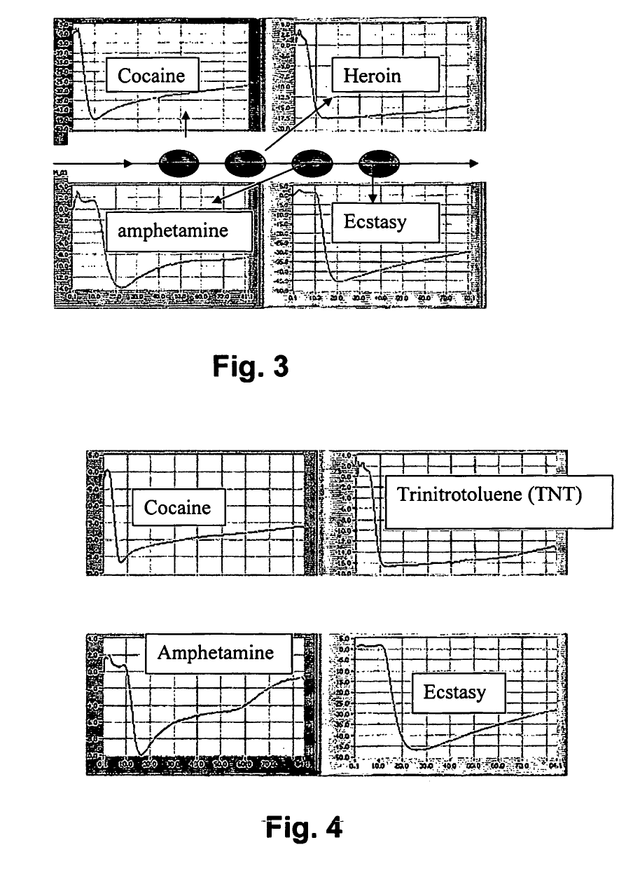

A negative frequency shift of 5-200 Hz of the piezoelectric crystal is observed during a short period, typically less than 20 seconds, in all the cells after the small injection of the MAB-mixture. (see FIGS. 3 and 4).

### DETAILED DESCRIPTION OF SOME DRAWINGS

FIG. 3. A typical response from four different biocells connected in series (See FIG. 2) upon a micro-injection of 4 microliters of antibody mixture and subsequent introduction of a blank sample at about 40 sec.

FIG. 4. A typical response from four different biocells connected in series (See FIG. 2) upon a micro-injection of at first the 4 microliter antibody mixture (compare FIG. 3), subsequent introduction of 2 ng of D/L amphetamine after 40 seconds. (The serial order is the same as in FIG. 3, i.e. cocaine in cell 1, heroin in cell 2, amphetamine in cell 3 and ecstasy (MDMA) in cell 4.)

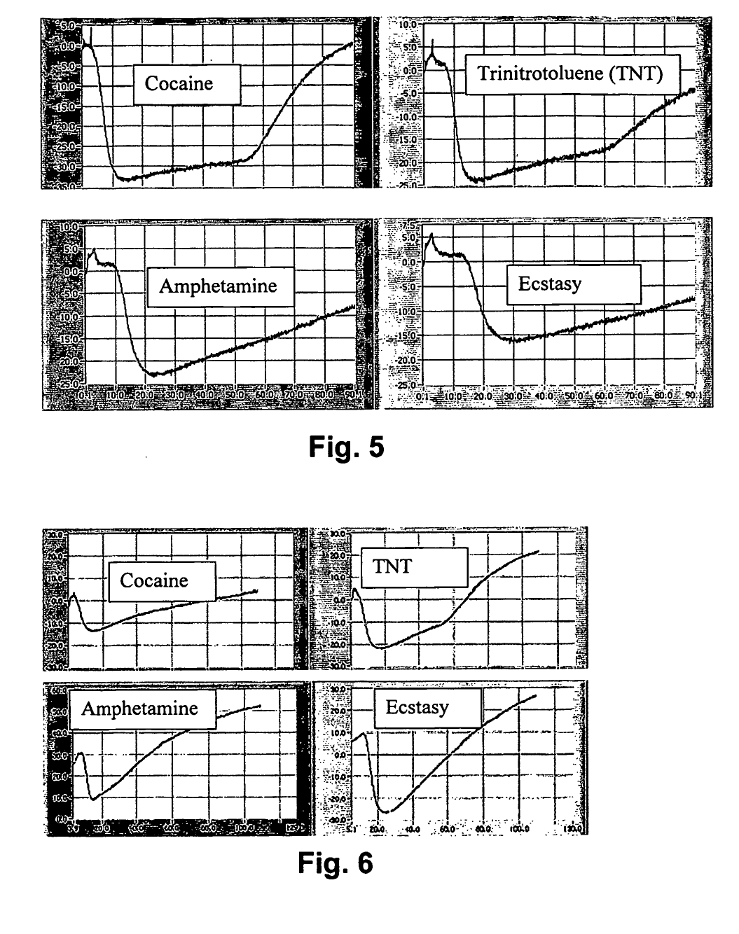

FIG. 5. A typical response from four different biocells connected in series (See FIG. 2) upon a micro-injection of at first the 4 microliter antibody mixture (compare FIGS. 3 and 4)), subsequent introduction of 2 ng of cocaine and 2 ng of TNT after 40 seconds. (The serial order is the same as in FIG. 3 apart from cell 2 , i.e. cocaine in cell 1, trinitrotoluene in cell 2, amphetamine in cell 3 and ecstasy (MDMA) in cell 4.)

FIG. 6. A typical response from four different biocells connected in series (See FIG. 2) upon a micro-injection of at first the 4 microliter antibody mixture (compare FIGS. 3 and 4 and 5), subsequent introduction of 2 ng of TNT after 40 seconds. (The serial order is the same as in FIG. 5., i.e. cocaine in cell 1, trinitrotoluene in cell 2, amphetamine in cell 3 and ecstasy (MDMA) in cell 4.)

The flow rate of the eluent, i.e. the buffer solution, in the instrument is kept constant between 10-200 microliters/minute depending on the desired dwell time in the cells, but can be different during the antibody activation step and during the sample analysis step in the analysis procedure.

We have concluded that it is most advantageous to modulate the affinity of the antibody to the antigen-analogue on the electrode in order to increase the sensitivity of the displacement reaction. The modulation is effected by adding chaotropic agents to the eluent, such as urea, guanidine hydrochloride, KSCN, MgClₛ various surfactants, e.g. Tween® 20 or Tween® 80, adjustment of pH to a value below or higher than 7 to accomplish a chaotropic effect etc.

The total analysis time, including antibody activation and analysis procedure (displacement), is less than 70 seconds (see FIG. 4). In automatic mode use of the multi flow cell piezoelectric crystal micro balance of our International patent application WO2004001392, the analysis time is approximately 40 seconds today.

The stability of the antibody mixture in the vial is normally several weeks or months at room temperature when stabilized.

A limited number of consecutive samples can be, in a repeatable way, injected after each MAB-activation. However, a MAB-activation, as illustrated in FIG. 4, before each introduction of a sample is necessary in order to maximize the sensitivity. As can be seen both in FIGS. 3 and 4, all the antigen-analogue coated QCM-electrodes are activated by means of the selective antibody, i.e. antibody specific for the analyte antigen. FIG. 4 also shows the response after introduction of a sample that contains one of the preselected antigens (2 ng of amphetamine). The positive response in the frequency in one of the cells is due to a selective displacement of the amphetamine antibody from only one of the electrodes, i.e. the only electrode that was prepared with amphetamine-analogue coating and activation with the antibody mixture containing an antibody specific for amphetamine as one of the antibodies. In the system, no cross-reaction is observed in any of the remaining cells which were similarly prepared with cocaine, heroin, and ecstasy (MDMA) analogues.

The stabilized mixture of at least two different antibodies, each specific for a predetermined antigen is particularly useful in a system for detection of several individual analytes in a test solution aliquot with an array of individually operated piezoelectric crystal microbalances, comprising flowing means for uninterrupted flowing of eluent solution, the antibody-mixture and the test solution aliquot to, and through, the cell compartment containing the piezoelectric crystal and measuring a change in oscillating characteristics of the crystal(s) following interaction between the antibody and the presence of the individual analytes in the test solution aliquot by the individually specific microbalances.

Further, the antibody-mixture is useful for simultaneously activation of a number of individual electrodes prior to an analysis.

Typically, a small volume, 1-50 microliters, e.g. 4 microliters, of a mixture of antibodies is introduced into the individual cells prior to the displacement analysis. This small volume passes through the cells in the order that they are fluidly connected to each other.

A commercial, preferably disposable, container, such as a vial, syringe, cassette or the like, typically contains the stable or stabilized mixture of antibodies for primary activation of several electrodes and for secondary, intermediate, activation during the run of several fluid samples in a screening situation.

An example of a vial containing a stabilized antibody mixture of the invention comprises in a total volume of 1 ml

0.1 mg/ml of each antigen-specific antibody
2 mg/ml of albumin
phosphate buffer of pH 7.4 in deionized water.

The stabilized antibody mixture is useful in a method of detecting an analyte in a fluid by using a sensor system, containing at least one oscillating piezoelectric crystal coated with specific antigens, introducing a small volume of specific antibodies followed by a fluid sample containing an analyte (antigen), and detecting change of mass on the coating on the piezoelectric crystal electrode as a change in oscillating characteristics resulting from interaction between the antigen and antibody.

To enhance the sensitivity of the method and the life time of the cells of the system, it is advantageous to add a detergent, such as 0.05% Tween® 20, to the eluent.

1. Mixture of isolated or synthetic affinity molecules in a liquid carrier comprising at least two different affinity molecules, each with affinity for a predetermined analyte, for use in a single or multi flow cell piezoelectric crystal micro balance apparatus.

2. Mixture according to claim 1, wherein each isolated or synthetic affinity molecule forms together with the predetermined analyte an interaction pair selected from the group consisting of anion-cation, antibody-antigen, receptor-ligand, enzyme-substrate, oligonucleotide-oligonucleotide with complementary sequence, oligonucleotide-protein, oligonucleotide-cell, and peptide nucleic acid (PNA) oligomer-polynucleotide, wherein the polynucleotide may be selected from the group consisting of RNA, DNA and PNA polymers complementary to the PNA oligomer.

3. Mixture according to claim 1, wherein each isolated or synthetic affinity molecule is selected from the group consisting of monospecific polyclonal or monoclonal antibodies, antibody fragments or derivatives thereof each with affinity for a predetermined analyte antigen.

4. Mixture according to claim 3, wherein the concentration of each of the different affinity molecules is between 0.01-0.8 mg/ml of the liquid carrier.

5. Mixture according to claim 1, wherein the liquid carrier is water and additionally contains a buffer, stabilizers and/or preservatives.

6. Mixture according to claim 1, wherein each of the analytes is selected from the group consisting of different narcotics selected from the group consisting of cocaine, heroin, amphetamine, methamphetamine, cannabinois, tetrahydrocannabinols (THC), and methylenedioxy-N-methylamphetamine (ecstacy).

7. Mixture according to claim 1, wherein each of the analytes is selected from the group consisting of different explosives selected from the group consisting of trinitrotoluene (TNT), dinitrotoluene (DNT), hexahydro-1,3,5-trinitro-1,3,5-triazine (RDX), octahydro-1,3,5,7-tetranitro-1,3,5,7-tetrazine (HMX), pentaerythritol tetranitrate (PETN), and nitroglycerine (NG).

8. Mixture according to claim 1, wherein each of the analytes is selected from the group consisting of different biomolecules, microorganisms and parts thereof.

9. Mixture according to claim 8, wherein the microorganisms are selected from bacteria, bacterial spores, mycobacteria, fungi, and viruses.

10. Use of a mixture according to claim 1 for introduction into the liquid flow of a single or multi flow cell piezoelectric crystal micro balance apparatus.

11. Use according to claim 10, wherein the mixture of affinity molecules is mixed with a test sample solution that possible contains one or several or the predetermined analyte(s) prior to introduction into the liquid flow of the apparatus for affinity binding competition with analyte-analogues of the predetermined analytes which analyte-analogues are immobilized on the electrode(s), for competition mode analysis.

12. Use according to claim 10, wherein the introduction into the liquid flow of the apparatus is for activation or reactivation of one or several flow cell crystal electrode(s) by attachment to analyte-analogues of the predetermined analytes which analyte-analogues are immobilized on the electrode(s).

13. Use according to claim 10, wherein the mixture is introduced into the continuous flow of the apparatus at intervals.

14. Use according to claim 13, wherein the interval is selected from the range of 20 minutes to 24 hours.

15. Use according to claim 13, wherein the mixture is introduced into the continuous flow of the apparatus after recovery of the electrode with an pH-lowering agent, such as glycine.

16. Kit containing a stable or stabilized mixture according to claim 1.
